(19) United States
(12) Patent Application Publication
(10) Pub. No.: US 2026/0294556 A1
(43) Pub. Date: Oct. 1, 2026

(54) ROBOT IN MRI-GUIDED INTERVENTIONS

(71) Applicant: Promaxo, Inc., Oakland, CA (US)

(72) Inventors: Akram Gholami PAREH, Oakland, CA (US); Neel SANCHETI, Milpitas, CA (US); Sreeja VANGAPELLI, Oakland, CA (US); Paul BRATHWAITE, Dublin, CA (US); Nariman Majdi NASAB, Oakland, CA (US); Aleksandar NACEV, San Francisco, CA (US)

(21) Appl. No.: 19/372,083

(22) Filed: Oct. 28, 2025

Related U.S. Application Data
(63) Continuation of application No. PCT/US2024/026640, filed on Apr. 26, 2024.
(60) Provisional application No. 63/462,914, filed on Apr. 28, 2023.

(51) Int. Cl. A61B 34/30; A61B 34/20; A61B 90/00

(52) U.S. Cl. CPC A61B 34/30; A61B 34/20; A61B 90/37; A61B 2034/2055; A61B 2090/374

(57) ABSTRACT

Disclosed herein are systems and methods relating to a new robot for MRI-guided interventions compatible with different MRI scanners, particularly low-field scanners with small bores. The systems and method disclosed herein may allow for precise, minimally invasive procedures.

### CROSS-REFERENCE

This application is a continuation of International Patent Application No. PCT/US 2024/026640, filed Apr. 26, 2024, which claims the benefit of U.S. Provisional Application No. 63/462,914, filed Apr. 28, 2023, the disclosure of which is incorporated herein by reference in its entirety.

### BACKGROUND

Magnetic resonance imaging (MRI) is an imaging modality that provides excellent soft tissue contrast, high spatial resolution, and multi-planar volumetric imaging capabilities.

### SUMMARY

In one aspect, a robotic system is provided. In some embodiments, the robotic system comprises: an end-effector configured to provide one or more percutaneous interventions; a base configured to fit inside a magnetic resonance scanner bore or be mounted to a bed; and a parallel manipulator configured to connect the end-effector to the base using a plurality of parallel kinematic chains.

In some embodiments, the parallel manipulator has three degrees of freedom (DOF).

In some embodiments, the one or more percutaneous interventions comprise biopsy or brachytherapy.

In some embodiments, the robotic system further comprises a plurality of pneumatic stepper motors configured to move the end-effector.

In some embodiments, the driving force of the plurality of pneumatic stepper motors comprises compressed air.

In some embodiments, at least one of the plurality of parallel kinematic chains comprises a proximal system connected to a distal system.

In some embodiments, the robotic system further comprises at least one sensor configured to recognize a home position of the end-effector.

In some embodiments, the robotic system further comprises at least one optical sensor.

In some embodiments, the at least one optical sensor is configured to track an output rotation of a plurality of pneumatic stepper motors.

In some embodiments, an additional one degree of freedom is added to the robotic system for driving an apparatus added to the end-effector.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “FIG.” and “FIG.” herein), of which:

FIG. 1 illustrates an isometric view of an exemplary MRI system, in accordance with some embodiments.

FIG. 2 illustrates a schematic of an MRI system, in accordance with some embodiments.

FIG. 3 illustrates a side view of exemplary shafts transferring the motion from actuators to kinematic chains, in accordance with some embodiments.

FIG. 4 illustrates a front view of exemplary pneumatic stepper motors, in accordance with some embodiments.

FIG. 5 illustrates a side view of an exemplary MRI system in an alternate configuration, in accordance with some embodiments.

FIG. 6 illustrates a perspective view of an exemplary robot inside a low-field MRI system, in accordance with some embodiments.

FIG. 7 illustrates a perspective view of an exemplary robot inside a low-field MRI system where a part of the scanner is cropped for a better view, in accordance with some embodiments.

### DETAILED DESCRIPTION

The present disclosure relates to medical devices. More particularly, this disclosure relates to a robotic system made of medical imaging-safe materials that can be utilized for performing MRI-guided diagnostic procedures and minimally invasive therapeutic interventions.

Magnetic resonance imaging (MRI) is an imaging modality that provides excellent soft tissue contrast, high spatial resolution, and multi-planar volumetric imaging capabilities. In some cases, a body-mounted robot with fluid-driven actuators to position needles for biopsy or RF ablation was developed. However, the system can use a manual placement on the patient and guided manual coarse targeting based on an initial MRI dataset. Also, the interventionalist may manually place the needle out-of-bore. Another system is an MRI-compatible surgical robot that can perform microsurgery and stereotaxy for brain and spine surgeries. The robot can use MRI imaging to guide the surgical instruments and provide real-time feedback to the surgeon. A parallel robot for prostate biopsies to track and steer needles to reach targets behind obstacles was built.

These systems can use MRI to guide surgical instruments, providing feedback to the surgeon and allowing for precise, minimally invasive procedures. This approach can reduce the risk of complications, shorten recovery times, and improve patient outcomes. Furthermore, these systems can decrease the time needed for procedures and minimize the need for additional diagnostic imaging, such as CT or ultrasound.

Image-guided robotic assistance may enhance clinical outcomes by facilitating more precise, less invasive, and more effective interventions, reducing procedure infection, and leveraging medical imaging-based feedback. Another advantage of MRI-guided robot-assisted interventions may be their potential to reduce procedure time. In standard intraoperative procedures, the patient may be moved out of the bore for intervention and back into the bore for imaging. However, robots designed to perform interventions within the MRI bore can perform simultaneous intervention and imaging, reducing time while enhancing outcomes. Additionally, MRI-guided robot-assisted interventions can significantly improve ergonomics. Manual interventions in the closed-bore scanner can often be challenging.

While MRI imaging provides high-quality visual information during interventions, it may have limitations for conventional and robot-assisted interventions. In some cases, the spatial constraints inside the MRI bore limit access to the patient during imaging, making it challenging to use robotic systems that need to fit in the residual space between the patient and the MRI. Moreover, the magnetic field may impede using metal-based materials for robotic systems.

From another perspective, the healthcare disparity between developed and developing countries may increase interest in low-field MRI, which can provide helpful diagnostic information while not producing high-quality images. However, MRI-guided intervention research may be conducted in double-doughnut magnets, which may be difficult to implement in low-field MRI scanners.

Disclosed herein are systems and methods relating to a new robot for MRI-guided interventions compatible with different MRI scanners, particularly low-field scanners with small bores. The systems and method disclosed herein may allow for precise, minimally invasive procedures.

Disclosed herein is a robotic system that can be adapted with several intervention medical devices. Such medical devices can include, but are not limited to, various end-effectors for different percutaneous interventions such as biopsy or brachytherapy. In some cases, a robot is configured for use with MRI devices, including low-field strength MRI scanners. The challenges for robot-assisted MRI-guided interventions can arise from electromagnetic compatibility due to the MRI environment and space constraints due to typical closed-bore geometry. Bidirectional MRI compatibility can ensure that neither the device nor the scanner affects the other's function.

The disclosed robot may be size accessible within closed-bore tunnel-shaped scanners. The robot can be constructed of materials compatible with the working environment associated with MRI, such as, but not limited to, non-magnetic and dielectric materials such as plastics (e.g., polymer, ceramics, porcelain, metal oxides, rubbers, and other similar materials), or any combination thereof.

The robot may use high torque and resolution pneumatic stepper motors for moving the end-effector. The driving force of these motors may be compressed air. Optical sensors may be used for tracking the motor output rotation. The present robot may be configured and arranged so that the robot can be placed and travel within the bore of an MRI scanner.

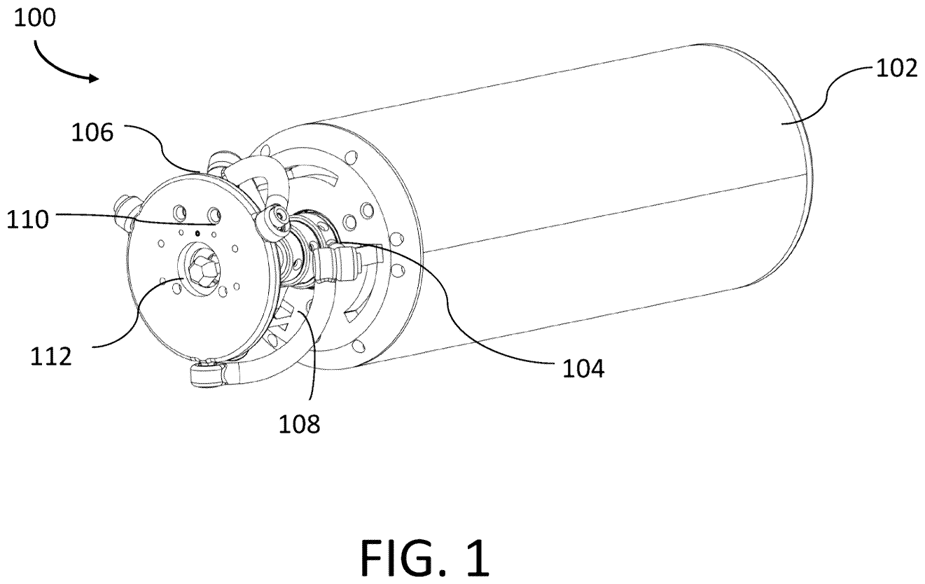

The developed robot may comprise a parallel manipulator with three degrees of freedom (DOF) in which the base platform of the robot is connected to a common plate/end-effector by three parallel identical kinematic chains, as shown in FIG. 1, in accordance with some embodiments. The manipulator 100 can comprise a base 102, three identical kinematic chains 104, and an end-effector 106. The robot may be at its home position when the end-effector 106 is parallel to the base 102. The base 102 can be designed to fit inside the scanner bore and can match the shape of the bore. Also shown in FIG. 1 is a sensor 108 for the home position of the end-effector 106, a geometric center 110 of manipulator 100, and a round Allen key mechanism 112.

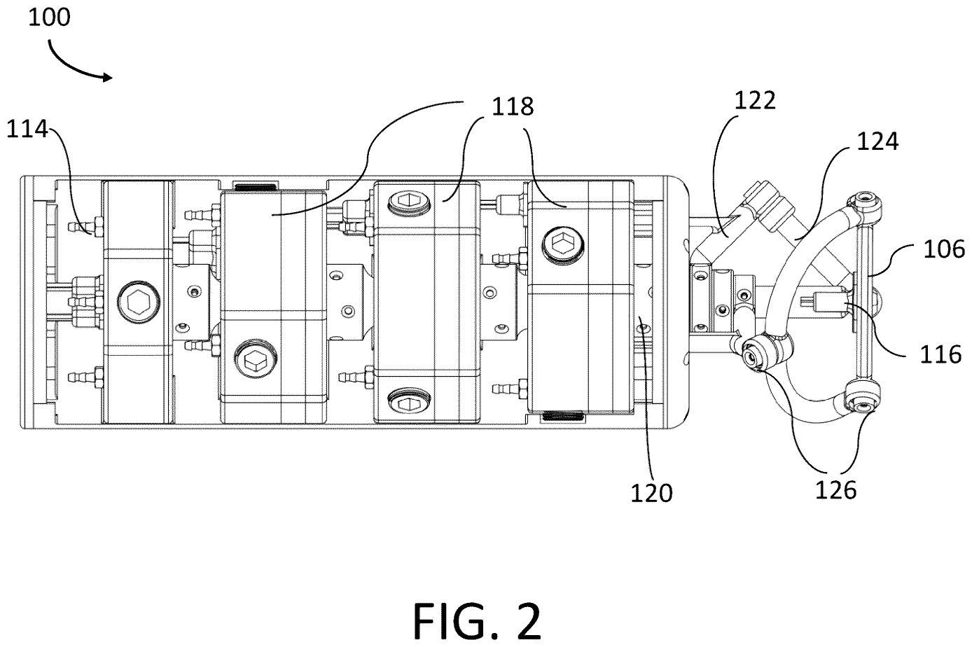

As shown in FIG. 2, each kinematics chain may comprise a proximal system 122 connected by a free revolute joint 126 to a distal system 124, in accordance with some embodiments. The top cover of the robot manipulator 100 is removed to show an internal view of the system. One main characteristic of the robot is that the base 102 comprises four collinear actuators. On each kinematics chain, one fixed actuated revolute joint 120 and two free revolute joints 126 may connect the proximal 122 and distal systems 124 with the end-effector 106. Three free revolute joints 126 may connect the distal systems 124 with the end-effector 106. Three rotary motors may actuate the proximal systems 122 on the base 102. This manipulator 100 can provide three rotational DOFs: roll, pitch, and yaw. FIG. 2 also shows air inlet 114, optical sensor 116, and high torque and resolution pneumatic stepper motors 118.

One key feature of this parallel manipulator can be that the axes of rotation of all the joints may intersect at a common point called the geometric center 110 of the manipulator 100. The geometric center 110 may be a point at which every end-effector element rotates about that point.

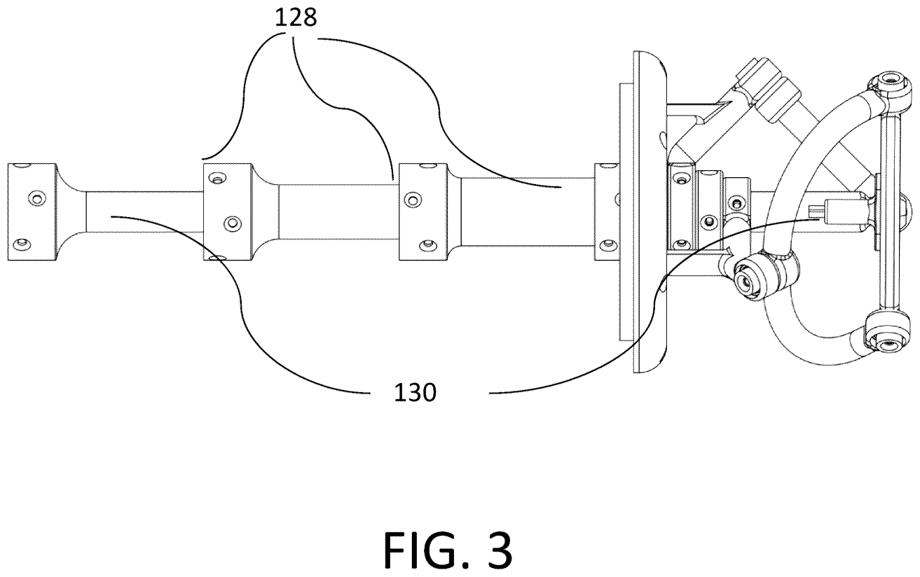

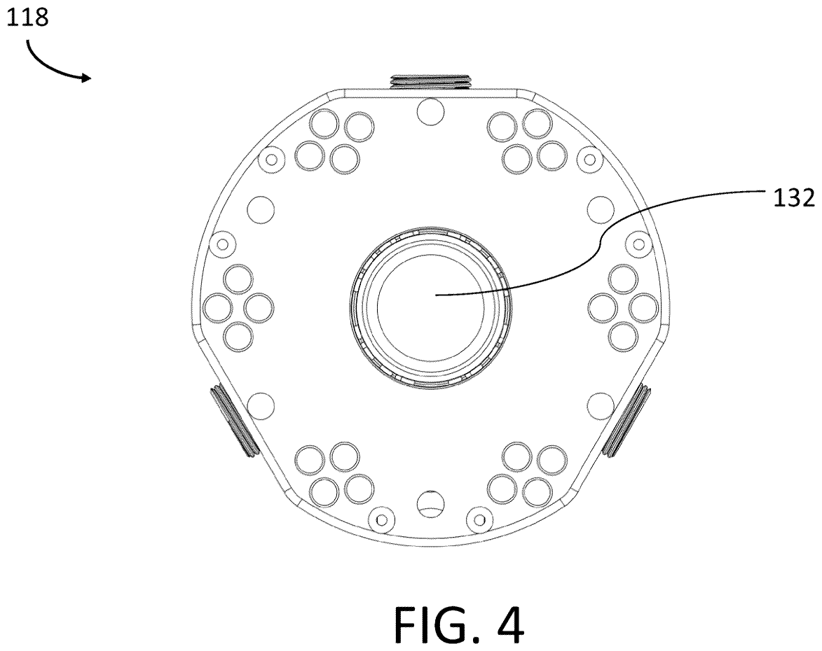

Another feature of the robot is that the base may contain four collinear actuators (FIG. 2). The shafts 128 transferring the motion from actuators to kinematic chains may be designed hollow to make the collinear actuator configuration possible (FIG. 3), in accordance with some embodiments. Referring to FIG. 3, in some cases, the shafts 128 transferring the motion from actuators to kinematic chains are designed to be hollow to make the collinear actuator configuration possible. That the actuator shaft 130 for driving any apparatus added to end-effector 106 may be also designed to be hollow. It should be noted that the center axis of the actuator of the high torque and resolution pneumatic stepper motors 118 may be designed to be hollow and/or may have a bore 132 (FIG. 4), in accordance with some embodiments.

The robot base may have three fiber optic sensors to home the proximal systems. When the three proximal systems rotate and each move in front of one optical sensor 116, the end-effector 106 may be parallel to the base. This position may be the home position for the robot.

An additional 1 DOF may be added to the system for driving any apparatus added to the end-effector. The output of the fourth actuator may be extended to the geometric center on the end-effector. This rotational movement on the end-effector can be transformed into any required movement. Since the end-effector can rotate about the roll, pitch, and yaw axis, a round-head Allen mechanism 112 may be configured to transfer the rotation to the apparatus (FIG. 1). The mechanism may comprise a cylindrical tool with a hexagonal shape at one end, which may fit into the socket of the driven part. A fiber optic sensor 116 can be placed on the end-effector to home the added apparatus.

The robot's base can be designed in different shapes and sizes to fit inside the scanner bore or be mounted on a patient bed. Adjusting the robot's location inside the bore can add another DOF to the system. Also, the shape of the end-effector can be modified to hold any required apparatus.

To drive the robot, a bundle of tubes, optic fibers, pneumatic air distributors, electrical circuits, a robot controller, or any combination thereof may be used. This equipment may be placed far from the magnetic fields. This equipment may operate reliably within the MRI system environment.

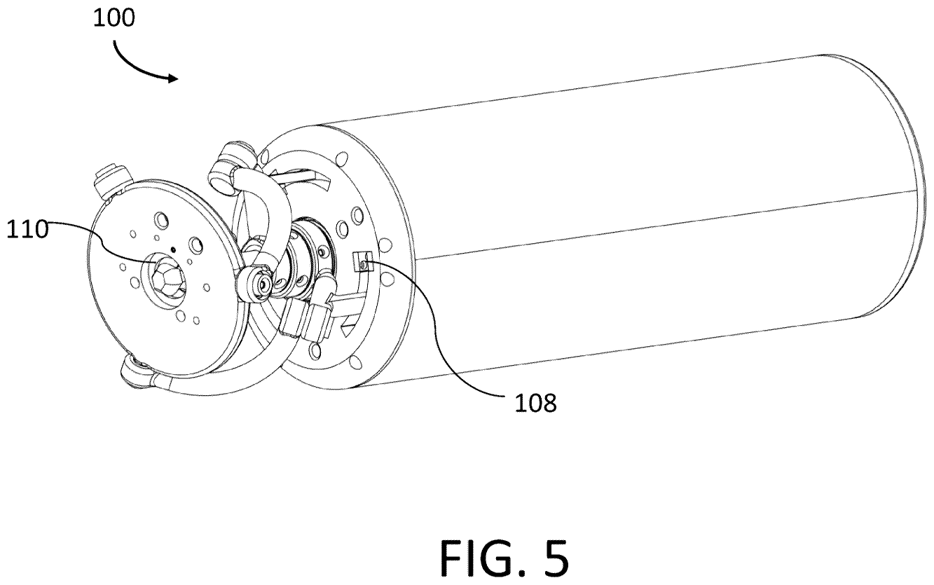

FIG. 5 illustrates a side view of robotic manipulator system 100 in an example random configuration other than a home position, in accordance with some embodiments. In some embodiments described herein, every element of the end-effector is rotating around the geometric center 110 of the robot. When the three proximal systems rotate and each move in front of one home positioning sensor 108 for home positioning of end-effector 106, the end-effector 106 may be parallel to the base. This position can be called the home position for the robot.

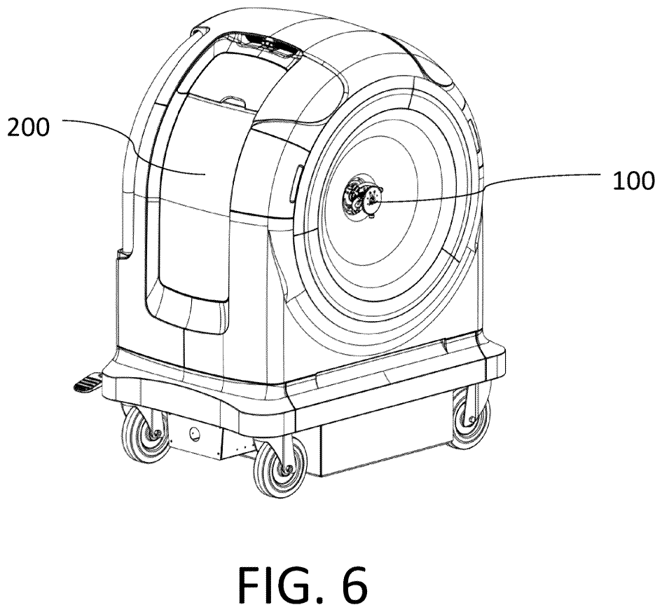

FIG. 6 illustrates a perspective view of the robot manipulator 100 inside a low-field MRI scanner system 200, in accordance with some embodiments. Since the robotic system shape is the same as the bore, it can move in the bore easily. It can also be fixed inside the bore.

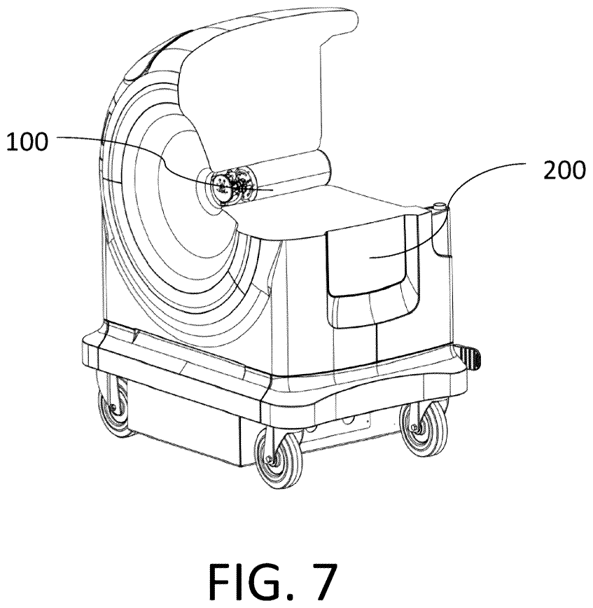

FIG. 7 illustrates a perspective view of the robot manipulator 100 inside a low-field MRI scanner system 200 where a part of the scanner is cropped for a better view, in accordance with some embodiments.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers, and also represent an amount close to the stated amount that still performs a desired function or achieves a desired result. The term “about” or “approximately” may mean within an acceptable error range for the particular value, which will depend in part on how the value is measured or determined, e.g., the limitations of the measurement system. For example, the terms “approximately”, “about”, and “substantially” may refer to an amount that is within less than 10% of, within less than 5% of, within less than 1% of, within less than 0.1% of, and within less than 0.01% of the stated amount. For example, “about” may mean within 1 or more than 1 standard deviation, per the practice in the art. Alternatively, “about” may mean a range of up to 20%, up to 10%, up to 5%, or up to 1% of a given value. As used herein, the term “about” a number refers to that number plus or minus 10% of that number. The term “about” a range refers to that range minus 10% of its lowest value and plus 10% of its greatest value. Where particular values are described in the application and claims, unless otherwise stated the term “about” meaning within an acceptable error range for the particular value may be assumed.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The section headings used herein are for organizational purposes only and are not to be construed as limiting the subject matter described.

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the present disclosure. It is intended that the following claims define the scope of the invention and that methods and structures within the scope of these claims and their equivalents be covered thereby.

1. A robotic system, comprising:
an end-effector configured to provide one or more percutaneous interventions;
a base configured to fit inside a magnetic resonance scanner bore or be mounted to a bed; and
a parallel manipulator configured to connect the end-effector to the base using a plurality of parallel kinematic chains.

2. The robotic system of claim 1, wherein the parallel manipulator has three degrees of freedom (DOF).

3. The robotic system of claim 1, wherein the one or more percutaneous interventions comprise biopsy or brachytherapy.

4. The robotic system of claim 1, further comprising a plurality of pneumatic stepper motors configured to move the end-effector.

5. The robotic system of claim 4, wherein a driving force of the plurality of pneumatic stepper motors comprises compressed air.

6. The robotic system of claim 1, wherein at least one of the plurality of parallel kinematic chains comprises a proximal system connected to a distal system.

7. The robotic system of claim 1, further comprising at least one sensor configured to recognize a home position of the end-effector.

8. The robotic system of claim 1, further comprising at least one optical sensor.

9. The robotic system of claim 8, wherein the at least one optical sensor is configured to track an output rotation of a plurality of pneumatic stepper motors.

10. The robotic system of claim 1, wherein an additional one degree of freedom is added to the robotic system for driving an apparatus added to the end-effector.

11. A method for performing MRI-guided diagnostic procedures, the method comprising:
a. MRI imaging a target area of a subject;
b. guiding, based at least in part on the MRI image, a robotic surgical system to the target area, the robotic surgical system comprising:
i. an end-effector configured to provide one or more percutaneous interventions;
ii. a base configured to fit inside a magnetic resonance scanner bore or be mounted to a bed; and
iii. a parallel manipulator configured to connect the end-effector to the base using a plurality of parallel kinematic chains;
c. providing substantially real-time feedback to the surgeon via the MRI image; and
d. performing a robotic surgical procedure via the robotic surgical system.

12. The method of claim 11, wherein guiding the robotic surgical system comprises simultaneously guiding the robotic surgical system and MRI imaging the target area.

13. The method of claim 11, wherein the parallel manipulator has three degrees of freedom (DOF).

14. The method of claim 11, wherein the one or more percutaneous interventions comprise biopsy or brachytherapy.

15. The method of claim 11, further comprising moving the end-effector via a plurality of pneumatic stepper motors.

16. The method of claim 15, wherein a driving force of the plurality of pneumatic stepper motors comprises compressed air.

17. The method of claim 15, wherein the robotic surgical system comprises at least one optical sensor; further comprising tracking an output rotation of the plurality of pneumatic stepper motors via the at least one optical sensor.

18. The method of claim 11, wherein at least one of the plurality of parallel kinematic chains comprises a proximal system coupled to a distal system via a free revolute joint.

19. The method of claim 11, further comprising recognizing a home position of the end-effector via at least one sensor.

20. The method of claim 11, wherein an additional one degree of freedom is added to the robotic system for driving an apparatus added to the end-effector.
